(19) United States
(12) Patent Application Publication
(10) Pub. No.: US 2019/0307364 A1
(43) Pub. Date: Oct. 10, 2019

(54) INTEGRATED DEVICE FOR LUNG HEALTH ASSESSMENT

(71) Applicant: The Regents of the University of California, Oakland, CA (US)

(72) Inventors: Kim D. Lu, Irvine, CA (US); Dan M. Cooper, Irvine, CA (US); Donald R. Blake, Irvine, CA (US)

(21) Appl. No.: 16/377,996

(22) Filed: Apr. 8, 2019

Related U.S. Application Data
(60) Provisional application No. 62/654,839, filed on Apr. 9, 2018.

(51) Int. Cl. A61B 5/083; A61K 9/00; G01N 33/497; G01N 33/94; A61B 5/08; A61B 5/085

(52) U.S. Cl. CPC A61B 5/0836; A61K 9/0075; G01N 33/497; G01N 33/94; A61B 2503/06; A61B 5/085; G01N 2800/52; G01N 2800/122; A61B 5/082

(57) ABSTRACT

The present invention relates to simple and integrated devices for assessing lung health. In one embodiment, an all-in-one device that is able to measure lung function, ventilation, inflammation and breath identification of aerosol propellant for monitoring asthma inhaler effectiveness and compliance. In another embodiment, the device uses a gas sensor composed of a sintered metal oxide and/or solid electrolyte that detects HFA-134a through a change in electrical conductivity or a change in voltage when gases are adsorbed on the sensor's surface.

### CROSS-REFERENCE TO RELATED APPLICATIONS

The present application claims the benefit of priority under 35 U.S.C. § 119(e) of provisional application Ser. No. 62/654,839, filed Apr. 9, 2018, the contents of which are hereby incorporated by reference.

### FIELD OF THE INVENTION

The present disclosure relates to the medical field, and more specifically, handheld devices for assessing lung health.

### BACKGROUND OF THE INVENTION

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Ordinarily, there are often multiple devices and technologies that are needed for purposes of assessing lung health in children and adults with respiratory concerns. Additionally, there are few quantitative ways to measure if inhaler medications are being used effectively or as prescribed. Inhaler compliance, for example, is assessed by self-report or pharmacy records. All of this results in several disadvantages, limitations and short-comings. For example, most general practitioners, emergency departments do not assess lung health using current technologies due to cost, need for multiple devices, portability. Most frequently these assessments of lung health are only available in specialized physician offices such as pediatric pulmonologists. Additionally, assessments of inhaler compliance via self-report or pharmacy records are inaccurate and thus there are not readily accessible methods available to measure inhaler effectiveness or compliance.

Thus, there is a need in the art for novel and more effective devices and methods of assessing lung health.

### SUMMARY OF THE INVENTION

Various embodiments include a device, comprising an hydrofluoroalkane (HFA) detector integrated with an apparatus, wherein the apparatus measures pulmonary function, airway inflammation, and/or ventilation. In another embodiment, the HFA detector comprises a gas sensor. In another embodiment, the gas sensor comprises a sintered metal oxide and/or solid electrolyte that detects HFA-134a. In another embodiment, the HFA-134a is detected through a change in electrical conductivity or a change in voltage when gases are absorbed on the sensor's surface. In another embodiment, the device further comprises a spinometer. In another embodiment, the device further comprises detection of nitric oxide. In another embodiment, the device further comprises detection of aveolar CO2 waveform. In another embodiment, the device is a handheld device. In another embodiment, the device further comprises an asthma aerosol dispenser.

Other embodiments include a method of assessing lung health in a subject, comprising: providing a device comprising an hydrofluoroalkane (HFA) detector integrated with an apparatus, wherein the apparatus measures pulmonary function, airway inflammation, and/or ventilation, and using the device to assess lung health in the subject. In another embodiment, the subject is a child. In another embodiment, the subject is an adult. In another embodiment, the subject is the user of the device as a means for a patient to assess their own health and/or follow a treatment plan by their physician.

Other embodiments include a treatment regimen for a lung related condition or disease, comprising assessing a subject using a device comprising an HFA detector integrated with an apparatus, wherein the apparatus measures pulmonary function, airway inflammation, and/or ventilation, and treating the subject. In another embodiment, the lung related condition or disease is asthma. In another embodiment, the treatment regimen comprises a medicinal composition. In another embodiment, the treatment regimen comprises an asthma aerosol dispenser.

Other features and advantages of the invention will become apparent from the following detailed description, taken in conjunction with the accompanying drawings, which illustrate, by way of example, various embodiments of the invention.

### DETAILED DESCRIPTION

All references, publications, and patents cited herein are incorporated by reference in their entirety as though they are fully set forth. Unless defined otherwise, technical and scientific terms used herein have the same meaning as commonly understood by one of ordinary skill in the art to which this invention belongs. Hornyak, et al., Introduction to Nanoscience and Nanotechnology, CRC Press (2008); Singleton et al., Dictionary of Microbiology and Molecular Biology 3rd ed., J. Wiley & Sons (New York, N.Y. 2001); March, Advanced Organic Chemistry Reactions, Mechanisms and Structure 7th ed., J. Wiley & Sons (New York, N.Y. 2013); and Sambrook and Russel, Molecular Cloning: A Laboratory Manual 4th ed., Cold Spring Harbor Laboratory Press (Cold Spring Harbor, N.Y. 2012), provide one skilled in the art with a general guide to many of the terms used in the present application. One skilled in the art will recognize many methods and materials similar or equivalent to those described herein, which could be used in the practice of the present invention. Indeed, the present invention is in no way limited to the methods and materials described.

As used herein, the term “spirometry” refers to measure of lung resistance and flow of air inhaled and exhaled. It is used to measure degree of airway obstruction in chronic lung diseases such as asthma.

As used herein, the term “CO₂ slope” refers to efficiency of how well the lung functions to mix incoming and existing gas in the lung. In a healthy lung, these two sources of gas are mixed completely, and thus the concentration of CO₂ in the exhaled breath is stable. In contrast, in patients with active lung disease, the mixing ability to the lung is impaired and thus the concentration of CO₂ will change between the beginning and end of the breath. This can be used to quantify the degree of lung disease.

As used herein, the term “exhaled nitric oxide” refers to measurement of the level of nitric oxide gas in exhaled breath. Increased levels of nitric oxide is associated with airway inflammation.

As used herein, the term “HFA” refers to hydrofluoroalkane.

As described herein, the inventors developed a diagnostic, simple integrated, medical device for assessing lung health. In accordance with various embodiments herein, for example, an all-in-one device was developed that is able to measure lung function, ventilation, inflammation and breath identification of aerosol and/or drug propellant for monitoring asthma inhaler effectiveness and compliance.

In one embodiment, the present invention provides an all-in-one, portable and/or simple device that has the capacity to measure pulmonary function, airway inflammation, ventilation and/or levels of HFA-134a (indicating asthma inhaler drug levels) in the breath. In one embodiment, it allows for multiple non-invasive assessments of the key determinants of lung health in patients with chronic lung diseases (including asthma, COPD) to physicians allowing for a personalized approach to managing chronic lung diseases such as asthma or chronic obstructive pulmonary disease. In another embodiment, the device is a patient compliance and teaching tool to measure effectiveness of asthma inhaler technique which is difficult to perform without guidance. In another embodiment, the device can be used by patients themselves in their own home, for example. In another embodiment, the device can be used by patients themselves and easily transmitted digitally to their physician. In another embodiment, for example, the device can be used by patients to assess their own health and follow the treatment plan outlined by their physician, for example.

In one embodiment, the present invention provides a handheld device, comprising an HFA detector integrated with an apparatus, wherein the apparatus measures pulmonary function, airway inflammation, and/or ventilation. In another embodiment, the HFA detector comprises a gas sensor. In another embodiment, the gas sensor comprises a sintered metal oxide and/or solid electrolyte that detects HFA-134a. In another embodiment, the HFA-134a is detected through a change in electrical conductivity or a change in voltage when gases are absorbed on the sensor's surface. In another embodiment, the device further comprises a spinometer. In another embodiment, the device further comprises detection of nitric oxide. In another embodiment, the device further comprises detection of aveolar CO2 waveform.

In another embodiment, the present invention provides a method of assessing lung health in a subject, comprising providing a device comprising an HFA detector integrated with an apparatus, wherein the apparatus measures pulmonary function, airway inflammation, and/or ventilation, and using the device to assess lung health in the subject. In another embodiment, the subject is a child. In another embodiment, the subject is an adult. In another embodiment, the subject is the user of the device as a means for a patient to assess their own health and/or follow a treatment plan by their physician.

In another embodiment, the present invention provides an overall treatment regimen for a lung related condition or disease, comprising assessing a subject using a device comprising an HFA detector integrated with an apparatus, wherein the apparatus measures pulmonary function, airway inflammation, and/or ventilation, and treating the subject.

In accordance with various embodiments herein, the present invention provides an integrated device that assesses lung health for patients with chronic lung diseases such as asthma, by measuring in aerosols used in asthma medications in the exhaled breath, hydrofluoroalkanes (HFA). In various embodiments, the device can also combine with one or more components that measure pulmonary function, airway inflammation and ventilation. In another embodiment, the integrated device may also include a pump or aerosol dispenser as well, such as used in asthma medications, and/or the composition dispensed from the pump or aerosol dispenser. In one embodiment, the composition additionally comprises from about 10% to 90% of propellant in order to provide a suitable pressure within the aerosol dispenser. In another embodiment, the invention also provides a method of preparing a pump dispenser containing the spray composition comprising mixing the ingredients of the composition with or without liquid propellant and placing the mixed ingredients in a pump dispenser. In addition, in one embodiment, the invention provides a method of preparing an aerosol dispenser containing the spray composition of the invention comprising mixing the ingredients of the composition without propellant and charging the mixture together with propellant into an aerosol dispenser. The composition is preferably dispensed from the chosen dispenser in a metered dose. In another embodiment, the medicament can be any medicinal compound in the salt or base form or a combination of compounds which is stable on mixing with the other ingredients of the composition and effective on topical administration.

### EXAMPLES

### Example 1

### Generally

An all-in-one simple device will measure pulmonary function, airway inflammation, ventilation and levels of HFA-134a (asthma inhaler aerosol propellant) in the breath. It will allow for multiple assessment of lung health at one time to physicians allowing for a personalized approach to managing chronic lung diseases such as asthma or chronic obstructive pulmonary disease. The device is also an educational tool to measure effectiveness of asthma inhaler technique which is difficult to perform without guidance. Lastly, the device could be used by patients themselves to assess their own health and follow the treatment plan outlined by their physician.

### Example 2

### Advantages

A portable, and/or simple, all-in-one device would be more accessible to a broader group of health professionals including general pediatricians, family medicine physicians, internists, emergency department physicians, telemedicine physicians or providers. The majority of adults and children with chronic lung diseases are managed by general practitioners or providers. Currently the multiple technologies needed to assess lung health are available primarily in clinics of specialized physicians such as pulmonologists and allergists, requiring staff educated physicians such as pulmonologists and allergists, requiring staff educated in the various modalities. Measuring of asthma inhaler drug levels would allow for a more personalized approach, for example, to the management of chronic lung diseases. Improvement of compliance is associated with improved outcomes including decreased ED visits, symptoms.

### Example 3

### Combination of HFA Detector with Measurement of Pulmonary Function, Airway Inflammation, and Ventilation

The inventors developed a simple integrated device that assesses lung health for patients with chronic lung diseases such as asthma. The device can combine existing technologies that measure pulmonary function, airway inflammation and ventilation with a novel method to measure effectiveness and compliance of inhaled medications. In one embodiment, one can accurately quantify asthma inhaled medication (corticosteroids (ICS) and bronchodilators) use by measuring in the exhaled breath the biologically inactive aerosol propellant (hydrofluoroalkane HFA-134a). Despite well documented benefits of ICS treatment for asthma, suboptimal compliance to prescribed asthma inhalers ranges between 30% and 70%. Poor compliance has been associated with greater morbidity and worse asthma outcomes. At this point, there are few technologies available to measure compliance in a non-invasive and effective manner. In another embodiment, the device is a handheld and cost-effective device that uses a gas sensor composed of a sintered metal oxide and/or solid electrolyte that detects HFA-134a through a change in electrical conductivity or a change in voltage when gases are adsorbed on the sensor's surface. In another embodiment, the device combines the HFA detector with tools that measure pulmonary function (spirometer), airway inflammation (exhaled nitric oxide) and ventilation (alveolar CO2 waveform). In another embodiment, the device could be used for measurement of compliance as well as effectiveness of inhaled medications for several groups: 1) general practitioners or pulmonologists in clinic; 2) emergency department physicians; 3) patients. This has the potential to transform care of adults and children with asthma.

### Example 4

### Significance

Chronic lung disease is common, debilitating, and costly. In conditions such as asthma, there are few reliable biomarkers (like a blood test) that can diagnose the disease. In addition, disease symptoms are variable and dynamic, and influenced by a myriad of factors including environmental exposure, psychosocial stress, and medication compliance. As a consequence, physicians must rely on a variety of physiological signals to gauge critical therapeutic decisions. Not surprisingly, the treatment of chronic lung diseases in both children and adults remains suboptimal.

In accordance with various embodiments herein, the inventors created a simple integrated device to measure lung health in children and adults. Although there may be existing technologies that can independently measure key functional features of chronic lung disease, a missing link has been the inability to tie this data to reliable estimates of medication use. In one embodiment, the device would integrate several existing tools that measure pulmonary function, airway inflammation and ventilation with a novel approach to measuring the effectiveness and compliance of commonly used inhaled medications. Combining these technologies could transform current paradigms of chronic lung disease treatment.

Currently, in the United States, approximately 24.6 million people, including over 6 million children are affected by asthma. The burden of asthma is significant, resulting in approximately 1.6 million visits to the Emergency Department and 13.8 million missed school days among children. The annual health care cost of asthma was as high as $56 billion in 2007 including millions of potentially avoidable hospital/ED visits, many involving patients who aren't compliant with their medications. Unfortunately, there is no cure for asthma and management of this chronic disease includes close monitoring of lung function, symptoms and appropriate treatment. Inhaled steroids (ICS) are the mainstay of treatment and proven effective at preventing asthma attacks, decreasing airway inflammation and are reported to be used by nearly 40% of all adults and children with asthma. However, despite the well documented benefit of ICS treatment for asthma control, suboptimal compliance to prescribed asthma medications is common (between 30-70%) and is associated with adverse clinical outcomes including asthma exacerbations and hospitalizations. Failure to teach patients to use inhaled medications properly and/or to detect non-compliance not only increases healthcare costs, but also makes management of asthma challenging and can result in inappropriate dose escalation and adverse consequences.

### Example 5

### Innovation and Impact

In one embodiment, the present invention provides a device an all in one and/or portable device designed to monitor lung health using existing technologies in addition to a novel, minimally invasive way to gauge inhaler use by measuring in aerosols used in asthma medications in the exhaled breath, hydrofluoroalkanes (HFA). HFAs are the most commonly used aerosol propellant in inhaled medications and are detectable for at least 48 hours in the exhaled breath. Inhalation of pharmaceuticals is challenging because of the inefficacy of delivery mechanisms. Measurement of breath HFA could not only detect compliance but also effectiveness of inhaler technique and serve as an educational tool. A simple portable device will allow general practitioners, emergency department physicians, telemedicine physicians, and patients themselves to measure lung function, airway inflammation, ventilation and compliance, clinical information useful in making appropriate treatment decisions. Using novel semiconductor technologies (Taguchi sensor), the inventors developed a small device that can measure HFA levels in the breath. Testing reveals that the HFA signal is detectable with this sensor indicating that the proposed technology can be rapidly adapted for the handheld device for patients with lung disease such as asthma. This novel technology combined with other potential devices has the potential to transform care of adults and children with asthma or other chronic lung diseases.

The various methods and techniques described above provide a number of ways to carry out the invention. Of course, it is to be understood that not necessarily all objectives or advantages described may be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as may be taught or suggested herein. A variety of advantageous and disadvantageous alternatives are mentioned herein. It is to be understood that some preferred embodiments specifically include one, another, or several advantageous features, while others specifically exclude one, another, or several disadvantageous features, while still others specifically mitigate a present disadvantageous feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be mixed and matched by one of ordinary skill in this art to perform methods in accordance with principles described herein. Among the various elements, features, and steps, some will be specifically included and others specifically excluded in diverse embodiments.

Although the invention has been disclosed in the context of certain embodiments and examples, it will be understood by those skilled in the art that the embodiments of the invention extend beyond the specifically disclosed embodiments to other alternative embodiments and/or uses and modifications and equivalents thereof.

Many variations and alternative elements have been disclosed in embodiments of the present invention. Still further variations and alternate elements will be apparent to one of skill in the art. Among these variations, without limitation, are the selection of constituent modules for the inventive compositions, and the diseases and other clinical conditions that may be diagnosed, prognosed or treated therewith. Various embodiments of the invention can specifically include or exclude any of these variations or elements.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

In some embodiments, the terms “a,” “an,” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Variations on those preferred embodiments will become apparent to those of ordinary skill in the art upon reading the foregoing description. It is contemplated that skilled artisans can employ such variations as appropriate, and the invention can be practiced otherwise than specifically described herein. Accordingly, many embodiments of this invention include all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that can be employed can be within the scope of the invention. Thus, by way of example, but not of limitation, alternative configurations of the present invention can be utilized in accordance with the teachings herein. Accordingly, embodiments of the present invention are not limited to that precisely as shown and described.

What is claimed is:

1. A device, comprising:
an hydrofluoroalkane (HFA) detector integrated with an apparatus,
wherein the apparatus measures pulmonary function, airway inflammation, and/or ventilation.

2. The device of claim 1, wherein the HFA detector comprises a gas sensor.

3. The device of claim 2, wherein the gas sensor comprises a sintered metal oxide and/or solid electrolyte that detects HFA-134a.

4. The device of claim 3, wherein the HFA-134a is detected through a change in electrical conductivity or a change in voltage when gases are absorbed on the sensor's surface.

5. The device of claim 1, further comprising a spinometer.

6. The device of claim 1, further comprising detection of nitric oxide.

7. The device of claim 1, further comprising detection of aveolar CO₂ waveform.

8. The device of claim 1, wherein the device is a handheld device.

9. The device of claim 1, further comprising an asthma aerosol dispenser.

10. A method of assessing lung health in a subject, comprising:
providing a device comprising an hydrofluoroalkane (HFA) detector integrated with an apparatus, wherein the apparatus measures pulmonary function, airway inflammation, and/or ventilation; and
using the device to assess lung health in the subject.

11. The method of claim 10, wherein the subject is a child.

12. The method of claim 10, wherein the subject is an adult.

13. The method of claim 10, wherein the subject is the user of the device as a means for a patient to assess their own health and/or follow a treatment plan by their physician.

14. A treatment regimen for a lung related condition or disease, comprising:
assessing a subject using a device comprising an HFA detector integrated with an apparatus, wherein the apparatus measures pulmonary function, airway inflammation, and/or ventilation; and
treating the subject.

15. The treatment regimen of claim 14, wherein the lung related condition or disease is asthma.

16. The treatment regimen of claim 14, comprising a medicinal composition.

17. The treatment regimen of claim 14, comprising an asthma aerosol dispenser.
